(19) United States
(12) Patent Application Publication
(10) Pub. No.: US 2012/0114634 A1
(43) Pub. Date: May 10, 2012

(54) METHODS FOR INDUCING A SUSTAINED IMMUNE RESPONSE AGAINST A B-CELL IDIOTYPE USING AUTOLOGOUS ANTI-IDIOTYPIC VACCINES

(75) Inventors: ANGELOS M. STERGIOU, ATHENS (GR); FRANCIS E. O'DONNELL, JR., LONGBOAT KEY, FL (US); CARLOS SANTOS, TAMPA, FL (US)

(73) Assignee: BIOVEST INTERNATIONAL, INC., TAMPA, FL (US)

(21) Appl. No.: 13/301,168

(22) Filed: Nov. 21, 2011

Related U.S. Application Data
(63) Continuation of application No. 13/081,426, filed on Apr. 6, 2011, now abandoned.
Application No. 13/081,426 is a continuation of application No. PCT/US2009/059880, filed on Oct. 7, 2009.
(60) Provisional application No. 61/103,499, filed on Oct. 7, 2008.

(51) Int. Cl. A61K 39/395; A61P 35/00

(52) U.S. Cl. 424/130.1

(57) ABSTRACT

The present invention relates to methods of inducing and maintaining an immune response against a B-cell idiotype in a subject using an autologous anti-idiotypic vaccine. In one embodiment, the immune response is induced and maintained for treatment of a B-cell derived malignancy selected from among non-Hodgkin's lymphoma. Hodgkin's lymphoma, chronic lymphocytic leukemia, multiple myeloma, and mantle cell lymphoma.

### CROSS REFERENCE TO RELATED APPLICATIONS

This application is a continuation of U.S. patent application Ser. No. 13/081,426, filed Apr. 6, 2011, which is a continuation of International Application No. PCT/US2009/059880, filed Oct. 7, 2009, which claims the benefit of U.S. Provisional Application Ser. No. 61/103,499, filed Oct. 7, 2008, the disclosure of each of which is hereby incorporated by reference in its entirety, including all figures, tables, and amino acid or nucleic acid sequences.

### BACKGROUND OF THE INVENTION

Surgery, chemotherapy and radiation therapy are the mainstay of cancer treatment and management. Surgery and radiation therapy are typically used to achieve results locally, whereas chemotherapy exerts a more systemic effect. However, usually remaining cancer cells are able to divide, thereby leading to a relapse of the cancer. Accordingly, despite the use of combination chemotherapy to treat various types of cancers, a significant number of cancers remain incurable.

More recently, immunotherapy based techniques have been developed for the treatment of various cancers. The central premise underlying immunotherapy for cancer is the presence of antigens which are selectively or abundantly expressed or mutated in cancer cells. For example, active immunotherapy involves delivering an antigen associated with a cancer to a patient, such that the patient's immune system elicits an immune response against the antigen and consequently, against the cancer cells expressing the antigen. Passive immunotherapy, on the other hand, involves administering immune therapeutics such as, for example, an antibody which selectively binds an antigen expressed on a cancer cell.

### BRIEF SUMMARY OF THE INVENTION

In one aspect, the present invention provides methods for maintaining an immune response against a B-cell idiotype in a subject that has undergone an initial treatment with an autologous anti-idiotypic vaccine, the method comprising administering at least one booster dose of the autologous anti-idiotypic vaccine to the subject. Preferably, the initial treatment elicits both a cellular and immoral immune response against the B-cell idiotype in the subject. In some embodiments, the booster dose(s) of the autologous anti-idiotypic vaccine is administered at least about 20 months after the initial treatment (i.e., last vaccination).

In some embodiments, the booster dose(s) of the autologous anti-idiotypic vaccine is administered to the subject about 24 months to about 30 months after completion of the initial treatment. In some embodiments, the booster doses of the autologous anti-idiotypic vaccine are administered to the subject about 24 months to about 30 months after completion of the initial treatment and administered again in about 12 to about 18 months thereafter. In some embodiments, the booster doses of the autologous anti-idiotypic vaccine are administered to the subject about 24 months to about 30 months after completion of the initial treatment and administered again in about 12 to about 18 months thereafter, and periodically at about every 12 to 18 months thereafter.

In some embodiments, the initial treatment is for a B-cell derived malignancy, such as non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, and Burkitt lymphoma/leukemia.

In some embodiments, the autologous anti-idiotypic vaccine comprises an antigen associated with a B-cell derived malignancy in the subject, and wherein the antigen is produced by a hybridoma. In some embodiments, the hybridoma is produced by fusion of a cancerous B-cell obtained from the subject and a murine/human heterohybridoma myeloma cell, such as the K6H6/B5 cell line or 1D12 cell line. In some embodiments, the antigen-producing hybridoma is grown in a hollow-fiber bioreactor. The can then be collected from the hollow-fiber bioreactor and purified (e.g., by affinity chromatography) prior to administration to the subject.

Preferably, in both the initial treatment and the one or more booster doses, the purified antigen is conjugated to a carrier molecule, such as an immunogenic carrier protein (e.g., keyhole limpet hemocyanin (KLH)) or other immunogenic carrier molecule, prior to administration to the subject.

Preferably, in the initial treatment, the autologous anti-idiotypic vaccine is administered in conjunction with an effective amount of an adjuvant, such as granulocyte monocyte-colony stimulating factor (GM-CSF). In some embodiments, the one or more booster doses of the autologous anti-idiotypic vaccine are administered without an adjuvant.

The initial treatment with the autologous anti-idiotypic vaccine can comprise one or more administrations. Preferably, the initial treatment is a regimen comprising a plurality of administrations of the autologous anti-idiotypic vaccine. In some embodiments, the initial treatment comprises five administrations of the autologous anti-idiotypic vaccine over a period of about 6 months. In some embodiments, the autologous anti-idiotypic vaccine comprises an antigen associated with a B-cell derived malignancy in the subject, and a carrier molecule linked to the antigen, and the initial treatment comprises administration (e.g., subcutaneous) of 0.01 mg to about 100 mg of the autologous anti-idiotypic vaccine (day 1) and about 50 μg/m²/day to about 200 μg/m²/day granulocyte monocyte-colony stimulating factor (days 1-4) at about 1, 2, 3, 4, and 6 months. In some embodiments, the autologous anti-idiotypic vaccine comprises an antigen associated with a B-cell derived malignancy in the subject, and keyhole limpet hemocyanin linked to the antigen, and the initial treatment comprises administration (e.g., subcutaneous) of 0.5 mg of the autologous anti-idiotypic vaccine (day 1) and 100 μg/m²/day granulocyte monocyte-colony stimulating factor (days 1-4) at about 1, 2, 3, 4, and 6 months.

In some embodiments, the booster dose comprises about 0.01 mg to about 100 mg autologous anti-idiotypic vaccine per administration (e.g., subcutaneous). In some embodiments, the booster dose comprises about 0.5 mg autologous anti-idiotypic vaccine per administration (e.g., subcutaneous).

In some embodiments, the subject has undergone a different therapy (i.e., other than the autologous anti-idiotypic vaccine therapy) prior to the initial treatment, such as chemotherapy and/or immunotherapy. In some embodiments, the different therapy comprises therapy with a monoclonal antibody, such as rituximab, tositumomab, ibritumomab tiuxetan, or epratuzumab (see, for example, Cheson B. D. and J. P. Leonard, N. Engl. J. Med., 359(6):613-626 (2008)). In some embodiments, the different therapy comprises a radioimmunotherapy, such as ibritumomab tiuxetan. In some embodiments, the different therapy comprises a regimen of PACE (prednisone, doxorubicin, cyclophosphamide, and etoposide) or CHOP-R (cyclophosphamide, hydroxydaunrubicin, oncovin, prednisone/prednisolone, and rituximab). Preferably, the different therapy induces complete remission in the subject prior to the initial treatment. Preferably, the subject is in complete remission at the time of the initial treatment. Preferably, the subject is in complete remission at the time that each of the one or more booster doses is administered.

Another aspect of the invention provides a method for maintaining a sustained immune response against a B-cell idiotype in a subject, the method comprising: (a) administering an effective amount of an autologous anti-idiotypic vaccine to the subject; and (b) administering at least one booster dose of the autologous anti-idiotypic vaccine to the subject. Preferably, the administering of (a) induces an immune response against a B-cell idiotype in the subject. Preferably, the immune response comprises both a cellular and humoral immune response. In some embodiments, the administering of at least one booster dose of (b) is conducted at least about 20 months after the administering of (a). In some embodiments, the at least one booster dose of (b) is administered to the subject about 24 months to about 30 months after the administering of (a). In some embodiments, the at least one booster dose of (b) is administered to the subject about 24 months to about 30 months after the administering of (a), and administered again in about 12 to about 18 months thereafter. In some embodiments, the at least one booster dose of (b) is administered to the subject about 24 months to about 30 months after the administering of (a), and administered again in about 12 to about 18 months thereafter, and periodically at about every 12 to 18 months thereafter.

In some embodiments, the administering step of (a) is for treatment of a B-cell derived malignancy, such as non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, and Burkitt lymphoma/leukemia.

In some embodiments, the autologous anti-idiotypic vaccine administered in (a) and (b) comprises an antigen associated with a B-cell derived malignancy in the subject, and the antigen is produced by a hybridoma. In some embodiments, the hybridoma is produced by fusion of a cancerous B-cell obtained from the subject and a murine/human heterohybridoma myeloma cell, such as the K6H6/B5 cell line or 1D12 cell line. In some embodiments, the antigen-producing hybridoma is grown in a hollow-fiber bioreactor. The antigen can then be collected from the hollow-fiber bioreactor and purified (e.g. by affinity chromatography) prior to administration to the subject. Preferably, in the administering steps of (a) and (b), the purified antigen is linked to a carrier molecule such as an immunogenic carrier protein (e.g., KLH) prior to administration to the subject.

Preferably, in the administering step of (a), the autologous anti-idiotypic vaccine is administered to the subject in conjunction with an effective amount of an adjuvant, such as GM-CSF. In some embodiments, the one or more booster doses of (b) are administered without an adjuvant.

The administering step of (a) can comprise one or more administrations of the autologous anti-idiotypic vaccine. Preferably, the administering step of (a) is a regimen comprising a plurality of administrations of the autologous anti-idiotypic vaccine. In some embodiments, the administering step of (a) comprises five administrations of the autologous anti-idiotypic vaccine over a period of about 6 months. In some embodiments, the autologous anti-idiotypic vaccine comprises an antigen associated with a B-cell derived malignancy in the subject, and a carrier molecule linked to the antigen, and the initial treatment comprises administration (e.g., subcutaneous) of 0.01 mg to about 100 mg of the autologous anti-idiotypic vaccine (day 1) and 50 μg/m²/day to about 200 μg/m²/day granulocyte monocyte-colony stimulating factor (days 1-4) at about 1, 2, 3, 4, and 6 months. In some embodiments, the autologous anti-idiotypic vaccine comprises an antigen associated with a B-cell derived cancer in the subject, and keyhole limpet hemocyanin linked to the antigen, and wherein said administering of (a) comprises administration (e.g., subcutaneous) of 0.5 mg of the autologous anti-idiotypic vaccine (day 1) and 100 μg/m²/day granulocyte monocyte-colony stimulating factor (days 1-4) at about 1, 2, 3, 4, and 6 months. In some embodiments, the booster dose(s) of step (b) comprises 0.01 mg to about 100 mg autologous anti-idiotypic vaccine per administration (e.g., subcutaneous). In some embodiments, the booster dose(s) of (b) comprises about 0.5 mg autologous anti-idiotypic vaccine per administration (e.g., subcutaneous).

In some embodiments, the subject has undergone a different therapy (i.e., other than the autologous anti-idiotypic vaccine therapy) prior to the administering of step (a), such as chemotherapy and/or immunotherapy. In some embodiments, the different therapy comprises therapy with a monoclonal antibody, such as rituximab, tositumomab, ibritumomab tiuxetan, or epratuzumab. In some embodiments, the different therapy comprises a radioimmunotherapy, such as ibritumomab tiuxetan. In some embodiments, the different therapy comprises a regimen of PACE (prednisone, doxorubicin, cyclophosphamide, and etoposide) or CHOP-R (cyclophosphamide, hydroxydaunrubicin, oncovin, prednisone/prednisolone, and rituximab). Preferably, the different therapy induces complete remission in the subject prior to the administering step of (a). Preferably, the subject is in complete remission at the time of the administering of (a). Preferably, the subject is in complete remission at the time that each of the one or more booster doses is administered in (b).

Another aspect of the invention provides a method for maintaining an immune response against a B-cell idiotype in a subject, the method comprising: (a) administering an effective amount of an autologous anti-idiotypic vaccine to the subject such that an immune response against the B-cell idiotype is induced; (b) assessing an immune response to the autologous anti-idiotypic vaccine in the subject and determining whether the immune response against the vaccine has diminished; and (c) administering at least one booster dose of the autologous anti-idiotypic vaccine to the subject if the immune response against the vaccine is determined to have diminished. In some embodiments, assessing of the immune response to the autologous anti-idiotypic vaccine of (h) comprises assessing the immune response against the B-cell idiotype. In some embodiments, the autologous anti-idiotypic vaccine comprises an antigen associated with a B-cell derived cancer in the subject, wherein the antigen is linked to a carrier molecule, and wherein assessing of the immune response to the autologous anti-idiotypic vaccine of (b) comprises assessing the immune response to the carrier molecule. In some embodiments, assessing of the immune response to the autologous anti-idiotypic vaccine of (b) comprises both assessing the immune response against the B-cell idiotype and assessing the immune response against the carrier molecule. In some embodiments, the determining of (b) comprises comparing the immune response as assessed after the administering of (a) to a prior or subsequent assessment of the immune response in the subject. In some embodiments, assessing of the immune response to the autologous anti-idiotypic vaccine of (b) is carried out multiple times at uniform or non-uniform time intervals after the administering of (a), and wherein the determining of (b) comprises comparing two or more of the multiple assessments to determine whether the immune response to the autologous anti-idiotypic vaccine has diminished. In some embodiments, the at least one booster dose of (c) is administered to the subject, and wherein the method further comprises administering at least one additional booster dose of the autologous anti-idiotypic vaccine to the subject if the immune response to the autologous anti-idiotypic vaccine is determined to have diminished since the at least one booster dose of (c).

The present invention provides methods of treating various B-cell derived malignancies and, in particular, B-cell derived cancers, such as, for example, non-Hodgkin's lymphoma, Hodgkin's lymphoma, chronic lymphocytic leukemia, mantle cell lymphoma or multiple myeloma, using an autologous anti-idiotypic vaccine.

In one aspect of the present invention, a method of eliminating or substantially reducing non-Hodgkin's lymphoma in a subject is provided. The method includes administering an effective amount of an autologous anti-idiotypic tumor vaccine, thereby to eliminate or substantially reduce non-Hodgkin's lymphoma in the subject and re-administering an effective amount of the autologous anti-idiotypic tumor vaccine (as a booster dose), thereby to maintain the elimination or substantial reduction of non-Hodgkin's lymphoma (e.g., to achieve and maintain complete clinical remission (no clinically detectable signs of disease)). In some embodiments, the booster dose(s) of the autologous anti-idiotypic vaccine is administered at least about 20 months after the initial administration. In some embodiments, the booster dose(s) of the autologous anti-idiotypic vaccine is administered to the subject about 24 months to about 30 months after completion of the first administration. In some embodiments, the booster doses of the autologous anti-idiotypic vaccine are administered to the subject about 24 months to about 30 months after completion of the first administration and administered again in about 12 to about 18 months thereafter. In some embodiments, the booster doses of the autologous anti-idiotypic vaccine are administered to the subject about 24 months to about 30 months after completion of the first administration and administered again in about 12 to about 18 months thereafter, and periodically at about every 12 to 18 months thereafter.

In another aspect of the present invention, a method of eliminating or substantially reducing Hodgkin's lymphoma in a subject is provided. The method includes administering an effective amount of an autologous anti-idiotypic tumor vaccine, thereby to eliminate or substantially reduce Hodgkin's lymphoma in the subject, and re-administering an effective amount of the autologous anti-idiotypic tumor vaccine, thereby to maintain the elimination or substantial reduction of Hodgkin's lymphoma (e.g., to achieve and maintain complete clinical remission (no clinically detectable signs of disease)). In some embodiments, the booster dose(s) of the autologous anti-idiotypic vaccine is administered at least about 20 months after the initial administration. In some embodiments, the booster dose(s) of the autologous anti-idiotypic vaccine is administered to the subject about 24 months to about 30 months after completion of the first administration. In some embodiments, the booster doses of the autologous anti-idiotypic vaccine are administered to the subject about 24 months to about 30 months after completion of the first administration and administered again in about 12 to about 18 months thereafter. In some embodiments, the booster doses of the autologous anti-idiotypic vaccine are administered to the subject about 24 months to about 30 months after completion of the first administration and administered again in about 12 to about 18 months thereafter, and periodically at about every 12 to 18 months thereafter.

In yet another aspect of the present invention, a method of eliminating or substantially reducing chronic lymphocytic leukemia (CLL) in a subject is provided. The method includes administering an effective amount of an autologous anti-idiotypic tumor vaccine, thereby to eliminate or substantially reduce chronic lymphocytic leukemia in the subject, and re-administering an effective amount of the autologous anti-idiotypic tumor vaccine, thereby to maintain the elimination or substantial reduction of CLL (e.g., to achieve and maintain complete clinical remission (no clinically detectable signs of disease)). In some embodiments, the booster dose(s) of the autologous anti-idiotypic vaccine is administered at least about 20 months after the initial administration. In some embodiments, the booster dose(s) of the autologous anti-idiotypic vaccine is administered to the subject about 24 months to about 30 months after completion of the first administration. In some embodiments, the booster doses of the autologous anti-idiotypic vaccine are administered to the subject about 24 months to about 30 months after completion of the first administration and administered again in about 12 to about 18 months thereafter. In some embodiments, the booster doses of the autologous anti-idiotypic vaccine are administered to the subject about 24 months to about 30 months after completion of the first administration and administered again in about 12 to about 18 months thereafter, and periodically at about every 12 to 18 months thereafter.

In a further aspect of the present invention, a method of eliminating or substantially reducing mantle cell lymphoma in a subject is provided. The method includes administering an effective amount of an autologous anti-idiotypic tumor vaccine, thereby to eliminate or substantially reduce mantle cell lymphoma in the subject, and re-administering an effective amount of the autologous anti-idiotypic tumor vaccine, thereby to maintain the elimination or substantial reduction of mantle cell lymphoma (e.g., to achieve and maintain complete clinical remission (no clinically detectable signs of disease)). In some embodiments, the booster dose(s) of the autologous anti-idiotypic vaccine is administered at least about 20 months after the initial administration. In some embodiments, the booster dose(s) of the autologous anti-idiotypic vaccine is administered to the subject about 24 months to about 30 months after completion of the first administration. In some embodiments, the booster doses of the autologous anti-idiotypic vaccine are administered to the subject about 24 months to about 30 months after completion of the first administration and administered again in about 12 to about 18 months thereafter. In some embodiments, the booster doses of the autologous anti-idiotypic vaccine are administered to the subject about 24 months to about 30 months after completion of the first administration and administered again in about 12 to about 18 months thereafter, and periodically at about every 12 to 18 months thereafter.

In yet another aspect of the present invention, a method of eliminating or substantially reducing multiple myeloma in a subject is provided. The method includes administering an effective amount of an autologous anti-idiotypic tumor vaccine, thereby to eliminate or substantially reduce multiple myeloma in the subject, and re-administering an effective amount of the autologous anti-idiotypic tumor vaccine, thereby to maintain the elimination or substantial reduction of multiple myeloma (e.g., to achieve and maintain complete clinical remission (no clinically detectable signs of disease)). In some embodiments, the booster dose(s) of the autologous anti-idiotypic vaccine is administered at least about 20 months after the initial administration. In some embodiments, the booster dose(s) of the autologous anti-idiotypic vaccine is administered to the subject about 24 months to about 30 months after completion of the first administration. In some embodiments, the booster doses of the autologous anti-idiotypic vaccine are administered to the subject about 24 months to about 30 months after completion of the first administration and administered again in about 12 to about 18 months thereafter. In some embodiments, the booster doses of the autologous anti-idiotypic vaccine are administered to the subject about 24 months to about 30 months after completion of the first administration and administered again in about 12 to about 18 months thereafter, and periodically at about every 12 to 18 months thereafter.

In one or more aspects of the present invention, a method for eliminating or substantially reducing non-Hodgkin's lymphoma or Hodgkin's lymphoma or chronic lymphocytic leukemia, mantle cell lymphoma or multiple myeloma further includes administration of an effective amount of granulocyte-monocyte colony stimulating factor (GM-CSF). In some embodiments, GM-CSF is administered in conjunction with an autologous anti-idiotypic vaccine.

In another aspect of the present invention, a method for eliminating or substantially reducing a B-cell derived cancer selected from the group consisting of non-Hodgkin's lymphoma, Hodgkin's lymphoma, chronic lymphocytic leukemia, mantle cell lymphoma and multiple myeloma is provided. The method includes administering an effective amount of an autologous anti-idiotype anti-tumor vaccine in conjunction with granulocyte-monocyte colony stimulating factor to the subject, thereby to eliminate or substantially reduce the B-cell derived cancer, and re-administering an effective amount of the autologous anti-idiotype anti-tumor vaccine. In one embodiment, the autologous anti-idiotype anti-tumor vaccine is administered without granulocyte-monocyte colony stimulating factor.

### BRIEF DESCRIPTION OF THE FIGURE

FIG. 1 is a graph showing disease-free survival from date of first vaccination in a cohort of human subjects with indolent follicular Non-Hodgkin's Lymphoma (NHL) treated during their first complete remission (treatment arms: A=Control; B=BiovaxID autologous anti-idiotypic vaccine). Patients in treatment arm B received BiovaxID® autologous anti-idiotypic vaccine subcutaneously on day 1 and GM-CSF on days 1-4 of months 1, 2, 3, 4, and 6. Patients in treatment arm A (control arm) received KLH/GM-CSF as in arm B.

### DETAILED DESCRIPTION OF THE INVENTION

### Definitions

In order that the present disclosure may be more readily understood, certain terms are first defined. Additional definitions are set forth throughout the detailed description.

The terms “eliminating,” “substantially reducing,” “treating,” and “treatment,” as used herein, refer to therapeutic or preventative measures described herein. The methods of “eliminating or substantially reducing” employ administration to a subject having non-Hodgkin's lymphoma, Hodgkin's lymphoma, chronic lymphocytic leukemia, mantle cell lymphoma or multiple myeloma, an autologous anti-idiotypic vaccine, such as to prevent, cure, delay, reduce the severity of, or ameliorate one or more symptoms of non-Hodgkin's lymphoma, Hodgkin's lymphoma, chronic lymphocytic leukemia, mantle cell lymphoma or multiple myeloma disorder, thereby prolonging the survival of a subject beyond that expected in the absence of such treatment. In some embodiments, the term “eliminating” refers to a complete remission of a cancer, e.g., non-Hodgkin's lymphoma, Hodgkin's lymphoma, chronic lymphocytic leukemia, mantle cell lymphoma or multiple myeloma in a subject treated using the methods described herein.

The terms “B lymphocyte” and “B cell,” as used interchangeably herein, are intended to refer to any cell within the B cell lineage as early as B cell precursors, such as pre-B cells B220⁺ cells which have begun to rearrange Ig VH genes and up to mature B cells and even plasma cells such as, for example, plasma cells which are associated with multiple myeloma. The term “B-cell,” also includes a B-cell derived cancer stem cell, i.e., a stem cell which is capable of giving rise to non-Hodgkin's lymphoma, Hodgkin's lymphoma, chronic lymphocytic leukemia, mantle cell lymphoma or multiple myeloma. Such cells can be readily identified by one of ordinary skill in the art using standard techniques known in the art and those described herein.

The term “immune tolerance,” as used herein, refers to a condition in which an animal recognizes a particular cell or antigen(s) as self, which should be recognized as foreign. In other words, the animal's immune system fails to mount an immune response to a cell or antigen(s) because the antigen is recognized as self instead of foreign. For example, the animal fails to mount an immune response against an antigen which is specifically expressed on a cancer cell.

The terms “immunoglobulin” and “antibody” (used interchangeably herein) include a protein having a basic four-polypeptide chain structure consisting of two heavy and two light chains, said chains being stabilized, for example, by interchain disulfide bonds, which has the ability to specifically bind an antigen. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind an antigen. The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable,” based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions.” The “constant” domains of an antibody light chain are referred to interchangeably as “light chain constant regions,” “light chain constant domains,” “CL” regions or “CL” domains. The “constant” domains of an antibody heavy chain are referred to interchangeably as “heavy chain constant regions,” “heavy chain constant domains,” “CH” regions or “CH” domains). The “variable” domains of an antibody light chain are referred to interchangeably as “light chain variable regions,” “light chain variable domains,” “VL” regions or “VL” domains). The “variable” domains of an antibody heavy chain are referred to interchangeably as “heavy chain constant regions,” “heavy chain constant domains,” “VH” regions or “VH” domains).

Immunoglobulins or antibodies can exist in monomeric or polymeric form, for example, IgM antibodies which exist in pentameric form and/or IgA antibodies which exist in monomeric, dimeric or multimeric form. Other than “bispecific” or “bifunctional” immunoglobulins or antibodies, an immunoglobulin or antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, (1990) Clin. Exp. Immunol. 79:315-321; Kostelny et al., (1992) J. Immunol. 148:1547-1553.

The term “antigen-binding portion” of an antibody (or “antibody portion”) includes fragments of an antibody that retain the ability to specifically bind to an antigen (e.g., a B-cell specific antigen). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P. et al., (1993) Proc. Natl. Acad. Sci. USA 90:6444-6448; Poljak, R. J. et al. (1994) Structure 2:1121-1123). Still further, an antibody or antigen-binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M. et al., (1995) Human Antibodies and Hybridomas 6:93-101) and use of a cysteine residue, a marker peptide and a C-terminal polyhistidine tag to make bivalent and biotinylated scFv molecules (Kipriyanov, S. M. et al., (1994) Mol. Immunol., 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. Preferred antigen binding portions are complete domains or pairs of complete domains.

“Specific binding,” “specifically binds,” “selective binding,” and “selectively binds,” as used herein, mean that the compound, e.g., antibody or antigen-binding portion thereof, exhibits appreciable affinity for a particular antigen or epitope and, generally, does not exhibit significant cross-reactivity with other antigens and epitopes. “Appreciable” or preferred binding includes binding with an affinity of at least 10⁶, 10⁷, 10⁸, 10⁹ M⁻¹, or 10¹⁰ M⁻¹. Affinities greater than 10⁷ M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant cross-reactivity” is one that will not appreciably bind to an undesirable entity (e.g., an undesirable proteinaceous entity). For example, in one embodiment, an antibody or antigen-binding portion thereof, that specifically binds to a B-cell specific antigen, such as, for example, CD-20 or CD-22, will appreciably bind CD-20 or CD-22, but will not significantly react with other non-CD-20 or non-CD-22 proteins or peptides. Specific or selective binding can be determined according to any art-recognized means for determining such binding, including, for example, according to Scatchard analysis and/or competitive binding assays.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

The term “human antibody” includes antibodies having variable and constant regions corresponding to human germline immunoglobulin sequences as described by Kabat et al. (See Kabat, et al., (1991) Sequences of'proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In a preferred embodiment, these replacements are within the CDR regions as described in detail below.

The term “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor, L. D. et al., (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences (See Kabat E. A., et al., (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or backmutation or both.

An “isolated antibody” includes an antibody that is substantially free of other antibodies having different antigenic specificities (e.g., an isolated antibody that specifically binds a B-cell specific antigen and is substantially free of antibodies or antigen-binding portions thereof that specifically hind other antigens, including other B-cell antigens). An isolated antibody that specifically binds a B-cell specific antigen may bind the same antigen and/or antigen-like molecules from other species. Moreover, an isolated antibody may be substantially free of other cellular material and/or chemicals.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

The terms “idiotype,” “Id,” and “idiotypic determinant,” as used herein, refer to an epitope in the hypervariable region of an immunoglobulin. Typically, an idiotype or an epitope thereof is formed by the association of the hypervariable or complementarity determining regions (CDRs) of VH and VL domains.

The terms “anti-idiotypic” and “anti-Id,” refer to the binding of an antibody or antigen-binding portion thereof to one or more idiotypes.

The term “autologous anti-idiotypic vaccine” refers to a composition, the active ingredient of which is an immunogenic molecule capable of inducing an immune response against a B-cell idiotype derived from the same subject to which it is administered. In some embodiments, the immunogenic molecule in a vaccine used in the methods of the present invention is a normal product of a subject's B cells that happens to be expressed clonally on the cancer cells (e.g., cells derived from a Hodgkin's lymphoma or non-Hodgkin's lymphoma or chronic lymphocytic leukemia, mantle cell lymphoma or multiple myeloma) and serves as a unique a target for immune attack. In some embodiments, an “autologous anti-idiotypic vaccine,” is capable of eliciting an immune response against a B-cell idiotype derived from a subject having non-Hodgkin's lymphoma. In another embodiment, an “autologous anti-idiotypic vaccine,” is capable of eliciting an immune response against a B-cell idiotype derived from a subject having Hodgkin's lymphoma. In yet another embodiment, an “autologous anti-idiotypic vaccine,” is capable of eliciting an immune response against a B-cell idiotype derived from a subject having chronic lymphocytic leukemia. In a further embodiment, an “autologous anti-idiotypic vaccine,” is capable of eliciting an immune response against a B-cell idiotype derived from a subject having multiple myeloma. In a yet further embodiment, an “autologous anti-idiotypic vaccine,” is capable of eliciting an immune response against a B-cell idiotype derived from a subject having mantle cell lymphoma. In some embodiments of the present invention, an “autologous anti-idiotypic vaccine,” is used for the treatment of a B-cell derived cancer in combination with other immune therapeutics such as, for example, monoclonal antibodies that selectively bind B-cell specific antigens. In some embodiments, an “autologous anti-idiotypic vaccine” includes an antigen associated with a B-cell derived cancer in a subject (e.g., non-Hodgkin's lymphoma, Hodgkin's lymphoma, chronic lymphocytic leukemia, mantle cell lymphoma or multiple myeloma) linked to KLH (keyhole limpet hemocyanin, a carrier protein). In some embodiments of the present invention, an autologous anti-idiotypic vaccine is administered in conjunction with GM-CSF, and subsequently re-administered, as a booster, one or times with or without GM-CSF.

The term “granulocyte monocyte colony stimulating factor” or “GM-CSF” refers to a hematopoeitic growth factor that stimulates the development of committed progenitor cells to neutrophils and enhances the functional activities of neutrophils. It is produced in response to specific stimulation by a variety of cells including macrophages, fibroblasts, endothelial cells and hone marrow stroma. Either purified GM-CSF or recombinant GM-CSF, for example, recombinant human GM-CSF (R & D SYSTEMS, INC, Minneapolis, Minn.) or sargramostim (LEUKINE, BAYER HEALTHCARE Pharmaceuticals, Wayne, N.J.) can be used in the methods described herein.

The phrase “an effective amount of granulocyte monocyte colony stimulating factor” refers to an amount of granulocyte monocyte colony stimulating factor, which upon a single or multiple dose administration to a subject, induces or enhances an immune response in the subject (e.g., as an adjuvant). In some embodiments, 50 μg/m²/day to about 200 μg/m²/day (e.g., 100 μg/m²/day) granulocyte monocyte colony stimulating factor is administered to the subject. In some embodiments, “an effective amount of granulocyte monocyte colony stimulating factor” refers to a daily administration of 5 μg/kg of the granulocyte colony stimulating factor.

### Exemplary Disorders

Exemplary disorders which may be treated using the methods of the invention include B-cell derived malignancies and in particular, B-cell derived cancers such as, for example, non-Hodgkin's lymphoma. Hodgkin's lymphoma, chronic lymphocytic leukemia, mantle cell lymphoma and multiple myeloma. Additional B-cell derived cancers include, for example, B-cell prolymphocytic leukemia, lymphoplasmocytic leukemia, splenic marginal zone lymphoma, marginal zone lymphoma (extra-nodal and nodal), plasma cell neoplasms (e.g., plasma cell myeloma, plasmacytoma, monoclonal immunoglobulin deposition diseases, heavy chain diseases), and follicular lymphoma (e.g., Grades I, II, III, or IV).

In some embodiments, a malignancy treated using the methods of the present invention is a B-cell derived malignancy associated with the expression of one or more B-cell specific antigens such as, for example, CD3d, CD5, CD6, CD9, CD19, CD20, CD21, CD22, CD23, CD24, CD27, CD28, CD37, CD38, CD40, CD45, CD46, CD48, CD53, CD69, CD70, CD72, CD73, CD79a, CD79b, CD80, CD81, CD83, CD85a, CD85d, CD85e, CD85h. CD85i, CD85j, CD85k, CD86, CD96, CD98, CD100, CD121b, CD124, CD127, CD132, CD150, CD152, CD154, CD157, CD166, CD169, CD179a, CD179b, CD180. CD185, CD196, CD197, CD205, CDw210a, CD213a1, CD257. CD267, CD268, CD269, CD274, CD275, CD276, CD278, CD279, CD300a, CD300c, CD307, CD314, CD316, CD317, CD319, CD320, CDw327, and CD331. In a particular embodiment, a cancer treated using the methods of the invention is associated with the expression of CD-20. In another embodiment, a cancer treated using the methods of the invention is associated with the expression of CD-22. In yet another embodiment, a cancer treated using the methods of the invention is associated with the expression of both CD-20 and CD-22.

In some embodiments, a cancer treated using the methods of the invention is non-Hodgkin's lymphoma or NHL. Non-Hodgkin's lymphoma or NHL, is a cancer of the lymphoid tissue which is formed by several types of immune cells including B-cells and T-cells. About 85% of the non-Hodgkin's lymphomas are derived from B-cells. NHL is thought to occur when B-cells, which produce antibodies, begin to grow abnormally. In some embodiments, non-Hodgkin's lymphoma treated using the methods of the invention is associated with the expression of CD-20 on B-cells. In other embodiments, non-Hodgkin's lymphoma is associated with the expression of CD-22. In yet other embodiments, non-Hodgkin's lymphoma is associated with the expression of both CD-20 and CD-22.

In some embodiments, a cancer treated using the methods of the invention is Hodgkin's lymphoma, also referred to as Hodgkin's disease. The cancer cells in Hodgkin's disease are called Reed-Sternberg cells, after the two doctors who first described them in detail. Under a microscope they look different from cells of non-Hodgkin's lymphomas and other cancers, and are believed to be a type of malignant. B lymphocyte.

In some embodiments, a cancer treated using the methods of the invention is chronic lymphocytic leukemia (CLL) which is derived from a small B lymphocyte. CLL is mostly found in the blood and in the hone marrow.

In further embodiments, a cancer treated using the methods of the invention is mantle cell lymphoma.

In yet other embodiments, a cancer treated using the methods of the invention is multiple myeloma, associated with uncontrolled proliferation of antibody producing cells in the plasma, which develop from B-cells.

### Exemplary Autologous Anti-Idiotypic Vaccines

In various embodiments of the methods of the present invention, an autologous anti-idiotypic vaccine is produced using a hybridoma technology. For example, a hybridoma cell-line may be developed which contains a tumor-specific antigen derived from a patient, which is unique to that patient and found exclusively on the surface of a B-lymphocyte associated with a B-cell derived cancer such as, for example, non-Hodgkin's lymphoma, Hodgkin's lymphoma, chronic lymphocytic leukemia, mantle cell lymphoma or multiple myeloma, and which is absent or expressed in decreased amounts in normal B-lymphocytes and other cells.

In some embodiments, an “autologous anti-idiotypic vaccine” includes an antigen associated with a B-cell derived cancer in a subject (e.g., non-Hodgkin's lymphoma, Hodgkin's lymphoma, chronic lymphocytic leukemia, mantle cell lymphoma or multiple myeloma) linked to a carrier molecule, such as a carrier protein. Preferably, the carrier molecule is immunogenic, such as the immunogenic carrier protein KLH ((keyhole limpet hemocyanin) Kwak L W et al., N Engl. J. Med., 327:1209-1215 (1992); Hsu F J et al., Blood, 89:3129-3135 (1997); Schumacher K, J. Cancer Res. Clin. Oncol., 127(Suppl 2):R1-R2 (2001)). An exemplary autologous anti-idiotypic vaccine is BIOVAXID®.

In some embodiments, the autologous anti-idiotypic vaccine comprises an antigen associated with a B-cell derived malignancy in the subject, and wherein the antigen is produced by a hybridoma (see, for example, Lee S T et al., Expert Opin Biol Ther, 7(1):113-122 (2007); Flowers C R, Expert Rev Vaccines, 6(3):307-317 (2007); Neelapu S S and L W Kwak, Hematology, 243-249, (2007); Lee S-T. et al., Yonsei Medical Journal, 48(1):1-10 (2007); Ruffini P A et al., Haematologica, 87:989-1001 (2002), which are each incorporated herein by reference in their entirety). In some embodiments, the hybridoma is produced by fusion of a cancerous B-cell obtained from the subject and a murine/human heterohybridoma myeloma cell, such as the K6H6/B5 cell line or 1D12 cell line. In some embodiments, the antigen-producing hybridoma is grown in a hollow-fiber bioreactor, such as those described in one or more of International Patent Publications WO 2007/139748 (Biovest International, Inc., filed May 21, 2007); WO 2007/139742 (Biovest International, Inc., filed May 21, 2007); WO 2007/139746 (Biovest International, Inc., filed May 21, 2007); WO 2007/136821 (Biovest international, Inc., filed May 21, 2007); and WO 2007/139747 (Biovest International, Inc., filed May 21, 2007), each of which are incorporated herein by reference in their entirety). The antigen can then be collected from the hollow-fiber bioreactor and purified (e.g., by affinity chromatography) prior to administration to the subject.

Preferably, in both the initial treatment with the autologous anti-idiotypic vaccine and the one or more booster doses of the autologous anti-idiotypic vaccine, the purified antigen is conjugated to a carrier molecule, such as an immunogenic carrier protein (e.g., KLH), prior to administration to the subject.

### Exemplary Antibodies

In various methods of the present invention, malignancies derived from B-cells can be treated using a combination of an autologous anti-idiotypic vaccine with one or more other therapies, such as a monoclonal antibody. The combination therapy may be consecutive (e.g., antibody therapy followed by autologous anti-idiotypic vaccine therapy) or contemporaneous. In some embodiments, malignancies derived from B-cells can be treated using a combination of an autologous anti-idiotypic vaccine with a monoclonal antibody which selectively binds a B-cell specific antigen. Examples of monoclonal antibody therapies include rituximab, tositumomab, ibritumomab tiuxetan, epratuzumab alemtuzumab, (see, for example, Cheson B. D. and J. P. Leonard, N. Engl. J. Med., 359(6):613-626 (2008)). Preferably, in any subjects receiving any of the pan-B-cell immunoablative therapies (e.g., Rituxan, Bexxar, Zevalin), any booster administrations of the autologous anti-idiotypic vaccine are administered at least about one month after such immunoablative therapies, as it typically takes approximately 14-21 days for B-cell recovery.

In some embodiments of the present invention, an antibody is a monoclonal antibody that specifically binds CD-20 on a B-cell. In other embodiments, an antibody is a monoclonal antibody that specifically binds CD-22 on a B-cell. However, without wishing to be bound by theory, it is contemplated that a human or humanized monoclonal antibody that selectively binds any one of B-cell specific antigens CD3d, CD5, CD6, CD9, CD19, CD20, CD21, CD22, CD23, CD24, CD27, CD28, CD37, CD38, CD40, CD45, CD46, CD48, CD52, CD53, CD69, CD70, CD72, CD73, CD74, CD79a, CD79b, CD80, CD81, CD83, CD85a, CD85d, CD85e, CD85h, CD85i, CD85j, CD85k, CD86, CD96, CD98, CD100, CD121b, CD124, CD127, CD132, CD150, CD152, CD154, CD157, CD166, CD169, CD179a, CD179b, CD180, CD185, CD196, CD197, CD205, CDw210a, CD213a1, CD257, CD267, CD268, CD269, CD274, CD275, CD276, CD278, CD279, CD300a, CD300c, CD307, CD314, CD316, CD317, CD319, CD320, CDw327, CD331, Death receptor, or HLA-DR may be used in the methods of the invention.

Commercially available monoclonal antibodies that specifically bind B-cell specific antigens include, for example, rituximab, which binds CD-20, and epratuzumab, which binds CD-22 (see, for example, Cheson B. D. and J. P. Leonard, N. Engl. J. Med., 359(6):613-626 (2008)).

Antibodies or antigen-binding portions thereof can be tested for binding to a B-cell or a B-cell specific antigen by, for example, standard assays known in the art, such as ELISA, FACS analysis and/or Biacore analysis.

Antibodies or antigen-binding portions useful in the methods of the invention may be labeled with a detectable substance using well known techniques. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹⁴C, ¹²³I, ¹²⁴I, ¹²⁵I, ¹³¹I, ^{99m}Tc, ³⁵S or ³H.

### Modes of Administration

The various compounds used in the methods described herein may be administered orally, parenterally (e.g., intravenously), intramuscularly, sublingually, buccally, rectally, intranasally, intrabronchially, intrapulmonarily, intraperitonealy, topically, transdermally and subcutaneously, for example. The amount of compound administered in a single dose may dependent on the subject being treated, the subject's weight, the manner of administration and the judgment of the prescribing physician. Generally, however, administration and dosage and the duration of time for which a composition is administered will approximate that which are necessary to achieve a desired result.

In general, a therapeutically effective amount of a monoclonal antibody such as, for example, an antibody that specifically binds CD-20 or CD-22, from about 0.0001 mg/Kg to 0.001 mg/Kg; 0.001 mg/kg to about 10 mg/kg body weight or from about 0.02 mg/kg to about 5 mg/kg body weight. In some embodiments, a therapeutically effective amount of a monoclonal antibody is from about 0.001 mg to about 0.01 mg, about 0.01 mg to about 100 mg, or from about 100 mg to about 1000 mg, for example.

In some embodiments, a therapeutically effective amount of an autologous anti-idiotypic vaccine is from about 0.001 mg to about 0.01 mg, about 0.01 mg to about 100 mg, or from about 100 mg to about 1000 mg, for example. In some embodiments, an effective amount of the autologous anti-idiotypic vaccine is one or more doses of 0.5 mg.

In some embodiments, an effective amount of an antibody administered to a subject having -Hodgkin's lymphoma. Hodgkin's lymphoma, chronic lymphocytic leukemia or multiple myeloma between about 100 mg/m² and 200 mg/m², or between about 200 mg/m² and 300 mg/m² or between about 300 mg/m² and 400 mg/m². In a particular embodiment, an effective amount of a monoclonal antibody that selectively binds a B-cell specific antigen is about 375 mg/m².

The optimal pharmaceutical formulations for a desired monoclonal antibody can be readily determined by one or ordinary skilled in the art depending upon the route of administration and desired dosage. (See, for example, Remington's Pharmaceutical Sciences, 18th Ed. (1990), Mack Publishing Co., Easton, Pa., the entire disclosure of which is hereby incorporated by reference).

Antibodies for use in the methods or compositions described herein can be formulated for the most effective route of administration, including for example, oral, transdermal, sublingual, buccal, parenteral, rectal, intranasal, intrabronchial or intrapulmonary administration.

In some embodiment, the vaccine compositions used in the methods of the present invention include one or more cytokines such as, for example, GM-CSF. GM-CSF is a potent immunostimulatory cytokine with efficacy in promoting anti-tumor response, particularly T cell responses. In general, however, any cytokine or chemokine that induces inflammatory responses, recruits antigen presenting cells (APC) to the tumor and, possibly, promotes targeting of antigen presenting cells (APC) may be used in the vaccine compositions.

The autologous anti-idiotypic vaccines useful in the methods of the present invention may be administered by any conventional route including oral and parenteral. Examples of parenteral routes are subcutaneous, intradermal, transcutaneous, intravenous, intramuscular, intraorbital, intracapsular, intrathecal, intraspinal, intracisternal, intraperitoneal, etc. Preferably, the primary treatment and one or more booster doses of the autologous anti-idiotypic vaccine are administered by the same route, e.g., subcutaneously.

An effective amount of a vaccine composition administered to a subject will vary from individual to individual and can be, for example, between about 0.01 μg/kg and about 1 mg/kg body weight. The amount of the immunogen per dose can range from about 0.01 mg to 100 mg of protein per subject per injection.

Administration of the immunogenic (vaccine) composition is preferably by injection on one or multiple occasions to produce systemic immunity. In general, multiple administrations of the vaccine in a standard immunization protocol are used, as is standard in the art. For example, the vaccines can be administered at approximately two to six week intervals, or monthly, for a period of from one to six inoculations in order to provide protection. The vaccine may be administered by any conventional route including oral and parenteral. Examples of parenteral routes are subcutaneous, intradermal, transcutaneous, intravenous, intramuscular, intraorbital, intracapsular, intrathecal, intraspinal, intracisternal, intraperitoneal, etc.

Without wishing to be bound by theory, it is contemplated that vaccination may result in a systemic immune response, which includes either or both of an antibody response and a cell-mediated immune response, which will provide an anti-cancer therapeutic effect and/or result in antibodies and activated T lymphocytes of various classes which may be used themselves as therapeutic agents, for example, for producing passive immunity in cancer-bearing subjects.

The vaccine compositions used in the methods of the present invention may further include one or more adjuvants or immunostimulatory agents. Examples of adjuvants and immunostimulatory agents include, but are not limited to, aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, whole organisms or subcellular fractions of the bacteria Propionobacterium acnes or Bordetella pertussis, polyribonucleotides, sodium alginate, lanolin, lysolecithin, vitamin A, saponin and saponin derivatives, liposomes, levamisole, DEAE-dextran, blocked copolymers or other synthetic adjuvants. Such adjuvants are readily commercially available.

Depending on the intended mode of administration, the compounds used in the methods described herein may be in the form of solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. Each dose may include an effective amount of a compound used in the methods described herein in combination with a pharmaceutically acceptable carrier and, in addition, may include other medicinal agents, pharmaceutical agents, carriers, adjuvants, diluents, etc.

Liquid pharmaceutically administrable compositions can prepared, for example, by dissolving, dispersing, etc., a compound for use in the methods described herein and optional pharmaceutical adjuvants in an excipient, such as, for example, water, saline aqueous dextrose, glycerol, ethanol, and the like, to thereby form a solution or suspension. For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; see, for example, Remington's Pharmaceutical Sciences, 18th Ed. (1990), Mack Publishing Co., Easton, Pa., the entire disclosure of which is hereby incorporated by reference).

### Methods of Treatment

Methods of treatment described herein encompass methods of eliminating or substantially reducing a B-cell derived malignancy such as, for example, non-Hodgkin's lymphoma, Hodgkin's lymphoma, chronic lymphocytic leukemia, mantle cell lymphoma and multiple myeloma.

In some embodiments, the B-cell derived malignancy to be treated is selected from among non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, and Burkitt lymphoma/leukemia.

A subject having non-Hodgkin's lymphoma, Hodgkin's lymphoma, chronic lymphocytic leukemia, mantle cell lymphoma or multiple myeloma can be diagnosed using standard techniques known in the art. For example, a diagnosis may be made by removing a part of a lymph node and examining the cells under a microscope. Biopsies may also be taken from other body tissues.

Subsequent to diagnosis, a subject having non-Hodgkin's lymphoma, Hodgkin's lymphoma, chronic lymphocytic leukemia, mantle cell lymphoma or multiple myeloma can be treated using methods of the invention.

In some embodiments, a subject having non-Hodgkin's lymphoma or Hodgkin's lymphoma or chronic lymphocytic leukemia, mantle cell lymphoma or multiple myeloma is administered an effective amount of an autologous anti-idiotypic vaccine, which may optionally be administered in conjunction with an effective amount of GM-CSF, followed by re-administration of the autologous anti-idiotype vaccine one or more times as a booster.

In some embodiments, a subject having non-Hodgkin's lymphoma or Hodgkin's lymphoma or chronic lymphocytic leukemia or mantle cell lymphoma or multiple myeloma is administered an autologous anti-idiotypic vaccine (optionally in conjunction with GM-CSF) and an effective amount of a monoclonal antibody which specifically binds a B-cell specific antigen, e.g., CD-20 or CD-22, followed by re-administration of the autologous anti-idiotype vaccine, without the monoclonal antibody, as a booster.

In some embodiments, the booster dose(s) of the autologous anti-idiotypic vaccine is administered at least about 20 months after the initial treatment (i.e., at least 20 months after last vaccination). In some embodiments, the booster dose(s) of the autologous anti-idiotypic vaccine is administered to the subject about 24 months to about 30 months after completion of the initial treatment (i.e., after last vaccination). In some embodiments, the booster doses of the autologous anti-idiotypic vaccine are administered to the subject about 24 months to about 30 months after completion of the initial treatment and administered again in about 12 to about 18 months thereafter. In some embodiments, the booster doses of the autologous anti-idiotypic vaccine are administered to the subject about 24 months to about 30 months after completion of the initial treatment and administered again in about 12 to about 18 months thereafter, and periodically at about every 12 to 18 months thereafter.

The initial treatment with the autologous anti-idiotypic vaccine can comprise one or more administrations. Preferably, the initial treatment is a regimen comprising a plurality of administrations of the autologous anti-idiotypic vaccine. In some embodiments, the initial treatment comprises five administrations of the autologous anti-idiotypic vaccine over a period of about 6 months. In some embodiments, the autologous anti-idiotypic vaccine comprises an antigen associated with a B-cell derived malignancy in the subject, and a carrier molecule linked to the antigen, and the initial treatment comprises administration (e.g., subcutaneous) of 0.01 mg to about 100 mg of the autologous anti-idiotypic vaccine (day 1) and about 50 μg/m²/day to about 200 μg/m²/day granulocyte monocyte-colony stimulating factor (days 1-4) at about 1, 2, 3, 4, and 6 months. In some embodiments, the autologous anti-idiotypic vaccine comprises an antigen associated with a B-cell derived malignancy in the subject, and keyhole limpet hemocyanin linked to the antigen, and the initial treatment comprises administration (e.g., subcutaneous) of 0.5 mg of the autologous anti-idiotypic vaccine (day 1) and 100 μg/m²/day granulocyte monocyte-colony stimulating factor (days 1-4) at about 1, 2, 3, 4, and 6 months.

In some embodiments, the booster dose comprises about 0.01 mg to about 100 mg autologous anti-idiotypic vaccine per administration (e.g., subcutaneous). In some embodiments, the booster dose comprises about 0.5 mg autologous anti-idiotypic vaccine per administration (e.g., subcutaneous).

In some embodiments, the subject has undergone a different therapy (i.e., other than the autologous anti-idiotypic vaccine therapy) prior to the initial treatment, such as chemotherapy and/or immunotherapy. In some embodiments, the different therapy comprises therapy with a monoclonal antibody, such as rituximab, tositumomab, ibritumomab tiuxetan, or epratuzumab (see, for example, Cheson B. D. and J. P. Leonard, N. Engl. J. Med. 359(6):613-626 (2008)). In some embodiments, the different therapy comprises a radioimmunotherapy, such as ibritumomab tiuxetan. In some embodiments, the different therapy comprises a regimen of PACE (prednisone, doxorubicin, cyclophosphamide, and etoposide) or CHOP-R (cyclophosphamide, hydroxydaunrubicin, oncovin, prednisone/prednisolone, and rituximab). Preferably, the different therapy induces complete remission in the subject prior to the initial treatment with the vaccine. Preferably, the subject is in complete remission at the time of the initial treatment with the vaccine. Preferably, the subject is in complete remission at the time that each of the one or more booster doses is administered.

### Assessing Immune Response

One aspect of the invention provides a method for maintaining an immune response against a B-cell idiotype in a subject, the method comprising: (a) administering an effective amount of an autologous anti-idiotypic vaccine to the subject such that an immune response against the B-cell idiotype is induced; (b) assessing an immune response to the autologous anti-idiotypic vaccine in the subject and determining whether the immune response against the vaccine has diminished (e.g., in character and/or extent); and (c) administering at least one booster dose of the autologous anti-idiotypic vaccine to the subject if the immune response against the vaccine is determined to have diminished. The steps of (b) and (c) can be carried out multiple times, as needed.

An assessment can be made of the nature and/or extent of the subject's immune response to the vaccine (e.g. cellular and/or humoral response) one or more times after the initial treatment with the vaccine. Preferably, an assessment of the subject's immune response is also made before the subject's initial treatment with the autologous anti-idiotype vaccine (e.g., to establish a control or base-line for comparison to a subsequent assessment or assessments post-treatment). The subject's immune response to the vaccine can also be monitored by making an assessment before and after each booster dose is given. The timing and frequency of booster doses can be at the physician's discretion, and/or can be dependent on the results of assessments of the subject's immune response to the vaccine. For example, if the immune response is considered to be diminished (e.g., reduced or impaired in character and/or extent) following one of these assessments (e.g., either through loss of antibody response and/or a reduction of tumor-reactive T-cells or cytokines), it would indicate that the subject lost some of the immune response against the B-cell idiotype and therefore lost some anti-tumor immunity induced by the first cycle of vaccination. The physician could therefore consider administering a booster dose (e.g., one or more booster injections) or series of booster doses to the subject.

When assessing the subject's immune response, the immune response against the B-cell idiotype is preferably assessed. However, the assessment can include an assessment of the subject's immune response against any component of the vaccine. For example, an assessment of the subject's immune response against the anti-idiotype, or against a carrier molecule (e.g., KLH), or against both, can be made.

The subject's immune response can be monitored by making multiple assessments after the initial treatment at uniform time intervals (e.g., every three months, every six months, every nine months, or annually) or at non-uniform time intervals. Monitoring of the subject's immune response to the vaccine can continue for a pre-determined period of time, for a time determined based on therapeutic outcome, or indefinitely. Preferably, the subject's immune response is monitored from a time period starting prior to initial vaccination and continuing for a period of at least five years, or indefinitely.

Typically, each assessment will involve obtaining an appropriate biological sample from the subject. The appropriate biological sample will depend upon the particular aspect of the subject's immune response to be assessed (e.g. depending upon the particular assay). For example, in some embodiments, the biological sample will be one or more specimens selected from among blood, peripheral blood mononuclear cells (PBMC), and B-cell derived tumor. Samples for assessments are taken at a time point appropriate to obtain information regarding the immune response at the time of interest. For example, a sample may be taken from the subject from a time prior to vaccination and additional samples may be taken from the subject periodically after vaccination to determine the nature and extent of the immune responses observed.

In some embodiments, assessment of the immune response includes assessment of one or more of the following aspects of the immune response: anti-idiotype (anti-Id) humoral responses; B-cell derived tumor-specific antibodies; tumor-reactive T-cell precursor frequencies (e.g., via an IFN-gamma response); biomarkers in the B-cell derived tumor that correlate with clinical outcome following autologous anti-idiotype vaccine therapy; and B-cell derived tumor-specific CD4+ and CD8+ T-cell responses.

Preferably, the immune response is assessed by conducting one or more humoral response assays and/or cellular response assays, such as those described by Neelapu et al. (Nature Medicine, 11(9):986-991 (2005)), which is incorporated herein by reference in its entirety. Peripheral blood B and T cells can be collected from the subject and blood counts can be deteiinined, including but not limited to CD3−CD19+ B cells, CD3+CD4+ T cells, and CD3+CD8+ T cells. Tumor cells can be determined, and PBMCs isolated. Both B-cells and tumor cells can be activated with recombinant CD40 ligand trimer, as described in Neelapu et al. (2005). Depending on the type of immune response to be assessed (e.g., humoral, cellular, or both), one or more of the following assays may be used:
Humoral immune response assay: to assess anti-Id humoral responses and tumor-specific antibodies (see, for example, Kwak et al., Lancet. 345:1016-1020 (1995), which is incorporated herein by reference in its entirety).
IFN-gamma ELISPOT assay: to assess tumor-reactive T-cell precursor frequencies via an IFN-gamma response (see, for example, Malyguine et al., J. Trans. Med., 2:9 (2004) and Neelapu et al. Clin. Cancer Res., 10:8309-8317 (2004), which are each incorporated herein by reference in its entirety).
Cytokine induction assay: to assess biomarkers in the tumor that correlate with clinical outcome following autologous anti-idiotype vaccine therapy (see, for example. Neelapu et al. (2004)).
Intracellular cytokine assay: to assess tumor-specific CD4+ and CD8+ T-cell responses (Neealapu et al., J. Cancer Res. Clin. Oncol., 127 Suppl. 2, R14-19 (2001)).

Assays such as those listed above (either individually or in combination) can be used to periodically monitor (e.g., every 3, 6 months to 1 year) after a patient receives a course of the autologous anti-idiotypic vaccine, and may be used to determine an optimal schedule of booster vaccinations. In that case, if the immune response is considered to be reduced or impaired following one of these periodic tests (e.g., either through loss of antibody response and/or a reduction of tumor-reactive T-cells or cytokines), then the subject would be considered to have lost some of the anti-tumor immunity induced by the first cycle of vaccination. The physician could therefore consider administering a booster injection or series of injections to the subject.

The invention will be further described in the following examples, which are not meant to limit the scope of the invention in any way.

### Example 1

### Autologous Anti-Idiotypic Vaccine Prolongs Cancer-Free Survival

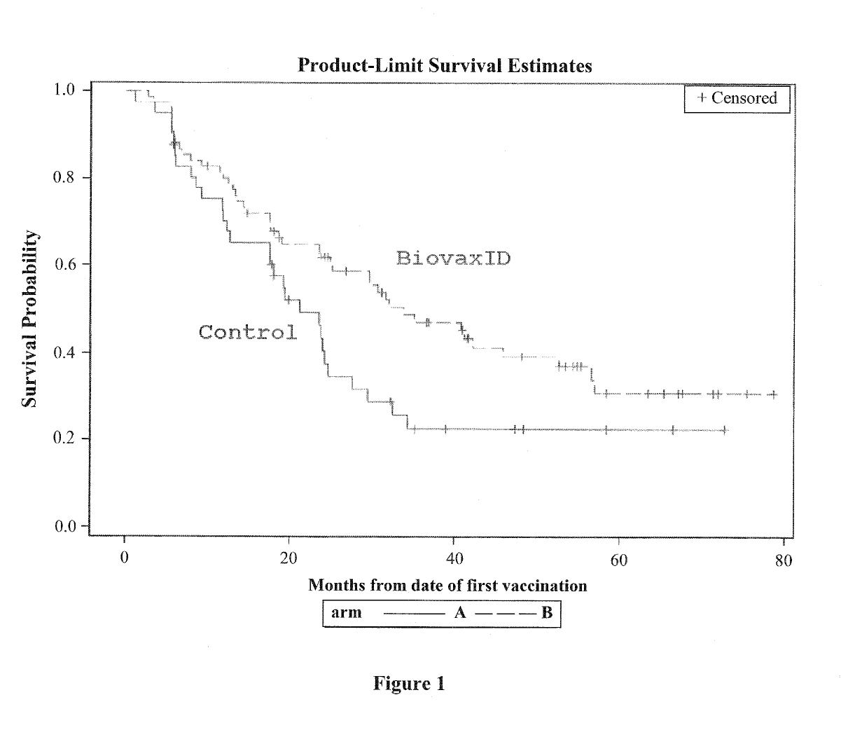

FIG. 1 is a graph showing disease-free survival from date of first vaccination with BiovaxID® autologous anti-idiotypic vaccine in a cohort of human subjects with indolent follicular Non-Hodgkin's Lymphoma (NHL) treated during their first complete remission. Patients with Stage III-IV follicular lymphoma and tumor>2 cm (Stage II allowed if tumor>5 cm), previously untreated by other than local radiation, provided tumor material by tissue biopsy for production of a patient-specific Ig idiotype vaccine conjugated to the immunogenic protein keyhole limpet hemocyanin (KLH). After completing PACE or CHOP-R chemotherapy and achieving a complete remission, followed by a waiting period to reconstitute the immune system, patients who remain in remission randomized to the active treatment arm received a series of 5 idiotype vaccinations (ID-KLH (0.5 mg subcutaneously)) at day 1, accompanied by the immune stimulant GM-CSF (100 μg/m²/day subcutaneously) at days 1-4 over a 6-month period at 1, 2, 3, 4, and 6 months time points. Patients randomized to the control arm received a time-matched series of KLH injections also accompanied by GM-CSF. Patients were subsequently studied to observe their immune responses both to the non-specific immune stimulating agents and for the specific immune response to the vaccine.

Enrollment was for newly diagnosed patients with follicular NHL, an often fatal blood cancer. Randomization required that patients achieve a complete clinical remission (CR or CRu) following chemotherapy. Both arms of the clinical trial are well-balanced in terms of stage and degree of malignancy and in terms of patient characteristics at enrollment and randomization. The intent-to-treat (ITT) analysis from the point of randomization for all patients in the trial who received at least one dose of BiovaxID® or control vaccination (n=117; 2:1 ratio of BiovaxID® versus control) showed that the median duration of complete remission in the BiovaxiD® arm of the study was 44.2 months which is clinically and statistically significant compared to the control arm, median duration of cancer-free survival of 30.6 months. BiovaxID® prolonged the cancer-free survival by 13.6 months or 44% (p-value=0.045; HR=1.6) with a median follow up of 56.6 months (range 12.6 to 89.3 months).

The time point at which the difference in disease-free survival between the two arms was greatest was approximately 36 months. At 36 months, 61% of BiovaxID® patients and 37% of control patients were cancer-free, meaning that BiovaxID® patients were 65% more likely to be cancer-free than were the control patients (p-value=0.023; HR=1.9). The data suggests that this may be an optimal time for supplemental booster shots, expected to further enhance the maintenance of clinical remissions.

Inclusion/exclusion criteria included diagnosis of indolent follicular lymphoma (follicular small-cleaved cell, follicular mixed or follicular large cell with centrocytes) with surface IgM or IgG phenotype; Stage III-IV with lymph node greater than 5 cm; no prior chemotherapy other than local radiation (not greater than 2 sites); ECOG less than 2; survival greater than 1 year; serum creatinine less than 1.5 mg/dl; bilirubin less than 1.5 mg/dl; SGOT/SGPT<3.5 ULN; no HIV antibodies or HBV antigen; negative pregnancy screen (females); no unrelated neoplasm in the previous 10 years; and no evidence of primary or secondary CNS lymphoma.

The specification is most thoroughly understood in light of the teachings of the references cited within the specification which are hereby incorporated by reference. The embodiments within the specification provide an illustration of embodiments in this disclosure and should not be construed to limit its scope. The skilled artisan readily recognizes that many other embodiments are encompassed by this invention. All publications and patents cited and sequences identified by accession or database reference numbers in this disclosure are incorporated by reference in their entirety. To the extent that the material incorporated by reference contradicts or is inconsistent with the present specification, the present specification will supercede any such material. The citation of any references herein is not an admission that such references are prior art to the present disclosure.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

What is claimed is:

1. A method for maintaining an immune response against a B-cell idiotype in a subject that has undergone an initial treatment with an autologous anti-idiotypic vaccine that elicited an immune response against the B-cell idiotype, the method comprising administering at least one booster dose of the autologous anti-idiotypic vaccine to the subject.

2. The method of claim 1, further comprising assessing an immune response to the autologous anti-idiotypic vaccine after the initial treatment.

3. The method of claim 2, wherein said assessing of the immune response to the autologous anti-idiotypic vaccine comprises assessing the immune response against the B-cell idiotype.

4. The method of claim 2, wherein said assessing of the immune response is carried out before said administering of at least one booster dose, after said administering of at least one booster dose, or before and after said administering of at least one booster dose.

5. The method of claim 2, further comprising comparing the immune response as assessed after the initial treatment to an assessment of the immune response in the subject carried out before the initial treatment.

6. The method of claim 2, wherein said assessing of the immune response to the autologous anti-idiotypic vaccine is carried out multiple times at uniform or non-uniform time intervals, and further comprising comparing two or more assessments to determine whether the immune response to the autologous anti-idiotypic vaccine has diminished.

7. The method of claim 6, further comprising administering at least one additional booster dose of the autologous anti-idiotypic vaccine to the subject if the immune response to the autologous anti-idiotypic vaccine is determined to have diminished.

8. The method of claim 1, wherein the at least one booster dose of the autologous anti-idiotypic vaccine is administered at least about 20 months after the initial treatment.

9. The method of claim 1, wherein the at least one booster dose of the autologous anti-idiotypic vaccine is administered to the subject about 24 months to about 30 months after completion of the initial treatment.

10. The method of claim 1, wherein at least one booster dose of the autologous anti-idiotypic vaccine is administered to the subject about 24 months to about 30 months after completion of the initial treatment and administered again in about 12 to about 18 months thereafter.

11. The method of claim 1 wherein at least one booster dose of the autologous anti-idiotypic vaccine is administered to the subject about 24 months to about 30 months after completion of the initial treatment and administered again in about 12 to about 18 months thereafter, and periodically at about every 12 to 18 months thereafter.

12. The method of claim 1, wherein the initial treatment is for treatment of a B-cell derived malignancy in the subject.

13. The method of claim 12, wherein the B-cell derived malignancy is selected from the group consisting of non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, and Burkitt lymphoma/leukemia.

14. The method of claim 12, further comprising assessing tumor response in the subject before the initial treatment, after the initial treatment, or before and after the initial treatment.

15. The method of claim 12, further comprising assessing tumor response in the subject before said administering of at least one booster dose, after said administering of at least one booster dose, or before and after said administering of at least one booster dose.

16. The method of claim 12, wherein the autologous anti-idiotypic vaccine comprises an antigen associated with a B-cell derived malignancy in the subject, and wherein the antigen is produced by a hybridoma.

17. The method of claim 16, wherein the hybridoma is produced by fusion of a cancerous B-cell obtained from the subject and a murine/human heterohybridoma myeloma cell, and wherein the murine/human heterohybridoma myeloma cell is the K6H6/B5 cell line or 1D12 cell line.

18. The method of claim 16, wherein the antigen-producing hybridoma is grown in a hollow-fiber bioreactor.

19. The method of claim 1, wherein the initial treatment is a regimen comprising a plurality of administrations of the autologous anti-idiotypic vaccine.

20. The method of claim 1, wherein the autologous anti-idiotypic vaccine comprises an antigen associated with a B-cell derived malignancy in the subject, and keyhole limpet hemocyanin linked to the antigen, and wherein the initial treatment comprises subcutaneous administration of 0.5 mg of the autologous anti-idiotypic vaccine (day 1) and 100 μg/m²/day granulocyte monocyte-colony stimulating factor (days 1-4) at about 1, 2, 3, 4, and 6 months.

21. The method of claim 1, wherein the at least one booster dose comprises about 0.01 mg to about 100 mg autologous anti-idiotypic vaccine per subcutaneous administration.

22. The method of claim 1, wherein the at least one booster dose comprises about 0.5 mg autologous anti-idiotypic vaccine per subcutaneous administration.

23. The method of claim 1, wherein the subject has undergone a different therapy prior to the initial treatment.

24. The method of claim 23, wherein the different therapy comprises chemotherapy and/or immunotherapy.

25. The method of claim 1, wherein the subject is in complete remission at the time of the initial treatment with the autologous anti-idiotypic vaccine.

26. The method of claim 1, wherein the subject is in complete remission at the time of said administering of at least one booster dose.

27. A method for inducing a sustained immune response against a B-cell idiotype in a subject, the method comprising:
(a) administering an effective amount of an autologous anti-idiotypic vaccine to the subject such that an immune response against the B-cell idiotype is induced; and
(b) administering at least one booster dose of the autologous anti-idiotypic vaccine to the subject such that the immune response against the B-cell idiotype is sustained.

28. The method of claim 27, further comprising assessing an immune response to the autologous anti-idiotypic vaccine after said administering of (a).

29. A method for maintaining an immune response against a B-cell idiotype in a subject, the method comprising:
(a) administering an effective amount of an autologous anti-idiotypic vaccine to the subject such that an immune response against the B-cell idiotype is induced;
(b) assessing an immune response to the autologous anti-idiotypic vaccine in the subject and determining whether the immune response against the vaccine has diminished; and
(c) administering at least one booster dose of the autologous anti-idiotypic vaccine to the subject if the immune response against the vaccine is determined to have diminished.

30. The method of claim 29, wherein said assessing of the immune response to the autologous anti-idiotypic vaccine of (b) is carried out multiple times at uniform or non-uniform time intervals after said administering of (a), and wherein said determining of (b) comprises comparing two or more of the multiple assessments to determine whether the immune response to the autologous anti-idiotypic vaccine has diminished.

31. The method of claim 29, wherein the at least one booster dose of (c) is administered to the subject, and wherein said method further comprises administering at least one additional booster dose of the autologous anti-idiotypic vaccine to the subject if the immune response to the autologous anti-idiotypic vaccine is determined to have diminished since the at least one booster dose of (c).
